(19) United States
(12) Patent Application Publication
(10) Pub. No.: US 2003/0068577 A1
(43) Pub. Date: Apr. 10, 2003

(54) Novel cyanine-TCNQ dye for high density data storage media

(76) Inventors: Wen-Yih Liao, Taichung (TW); Chien-Liang Huang, Taiwan (TW); Chuen-Fun Yan, Kao-Hsiung (TW); Huei-Wen Yang, Chung-Li City (TW); Tzuan-Ren Jeng, Hsin-Chu (TW); Der-Ray Huang, Hsin-Chu (TW); Andrew Teh Hu, Hsin-Chu (TW); Ming-Chia Lee, Tai-Chung Hsien (TW); Chung-Chun Lee, Yun-Ling Hsien (TW)

Correspondence Address: BACON & THOMAS, PLLC, 625 SLATERS LANE, FOURTH FLOOR, ALEXANDRIA, VA, 22314

(21) Appl. No.: 09/917,751

(22) Filed: Jul. 31, 2001

(30) Foreign Application Priority Data
May 17, 2001 (TW) 090111771

(51) Int. Cl. G11B 7/24; C07C 50/04

(52) U.S. Cl. 430/270.19; 430/270.21; 430/945; 428/64.8; 552/303

(57) ABSTRACT

This invention is to provide a kind of cyanine-TCNQ complex dyes mixture (II, III, and IV) used as the data storage media having the structural formula (I):

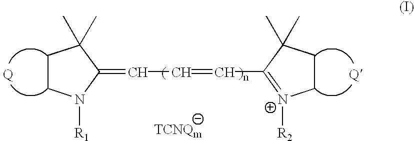

wherein Q and Q′ denote aromatic or polyaromatic, R1 and R2 are selected from the group consisting of alkyl, arylester, alkoxy, alkylthio, and alkoxythio etc., n represents an integer of 0, 1, 2, and 3, TCNQ-m represents 7,7′,8,8′-tetracyanoquinodimethane or its derivatives, and m represents an integer of 1 or 2. They can be used as the recording layer materials for the reflection optical recording media and the non-reflection fluorescent optical recording media after the suitable formulation.

### BACKGROUND OF INVENTION

1. Field of Invention

This invention is to provide a kind of cyanine-TCNQ complex dyes mixture (II, III, and IV), used as the data storage media, having the extremely large absorption and high fluorescent quantum efficiency in the visible light region with λ=400 nm˜800 nm, and can be used for the recording layer material of the reflection optical recording media and the non-reflection fluorescent optical recording media after the suitable formulation.

2. Description of the Prior Art

A storage medium with higher storage density, smaller size and lower cost is extremely required now-a-days. As magnetic storage medium is not able to satisfy these requirements, optical storage material is greatly sought for as an alternative. It is found that organic dye combines the advantages and characters of both the photosensitizer and photorecording medium, is highly developed and adopted for electronic industry. The applications of organic dye include in the fields of, for example, nonlinear optical device, recording and displaying of optical disc data, photoresist, sensor and indicator for heat, light, and electron, during the transfer and storage of energy, medicine and biology, etc.

Cyanine blue having cyanine structure was first synthesized by Greville Williams in 1856 (K. Venkataraman (ed.), The Chemistry of Synthetic Dyes, Vol. II, pp.1143-1186, Academic Press, New York, 1952). In 1875, Vogel discovered that cyanine blue has specific photosensitizing properties. Therefore, cyanine blue, which was originally used as dye for textile, turned to be a photosensitizer with the extremely higher added value. Cyanine dye covers in the range of photosensitizing from UV/Visible to IR according to the different structures. Therefore, cyanine dye can also serve as photosensitizer for photoresist or optical storage medium in electronic industry besides a photosensitizer for photo.

Cyanine TCNQ complex was first synthesized by J. H. Lupinski et al. in 1967. (“Tetracyanoquinodimethane Derivatives of Cyanine Dyes”, Vol. 3, pp.241-250, Molecular Crystals, Great Britain, 1967.) In 1981, 3,3′-diethyl- 12-acetyl-thiatetracyanine perchlorate was first proposed for application in optical disc fabrication by Law et al. (K. Y. Law, P. S. Vincett, and G. E. Johnson, Appl. Phys. Lett., 39, 718 (1981)). The cyanine dye and PVAc (poly(vinyl acetate)) were first mixed and then spin-coated to form an optical recording medium. After this successful application, several kinds of cyanine dye were developed for optical disc applications, such as those disclosed in U.S. Pat. Nos. 5,019,476, 5,292,615, 5,328,802, 5,332,608, 5,424,171, 5,455,094, and 5,579,150 etc.

In U.S. Pat. Nos. 5,019,476, 5,292,615, 5,328,802, 5,332,608, 5,424,171, and 5,455,094 an anion is one of the general common halide, PF6, and ClO4 etc.

Organic compounds containing the polymethine structure have the problem of the bad photostability. General cyanine dye also has the same problem since it belongs to this kind of compound. Hence, it is necessary to develop new type of cyanine dye having photostability. In 1999, Shin-ichi Morishima et al., Fuji-film company in Japan, attempted to use the doping method. (Shin-ichi Morishima et al., Jpn. J. Appl. Phys. Vol. 38 (1999), pp.1634-1637). Addition of neutral TCNQ derivative to dye increases the photostability of dye, however, ICNQ is not quite soluble in the common organic solvents so that content of doping dye formulation is limited, therefore, the stability effect is also limited.

### SUMMARY OF THE INVENTION

Hence, the aim of this invention is to solve the drawbacks described above. In order to avoid the presence of the drawbacks described above, this invention is to provide a kind of cyanine-TCNQ complex dyes mixture (II, III, and IV), used for the data storage media, with short wavelength (□00 nm˜400 nm) and near infrared region (800 nm˜1000 nm), having high absorption for light, resisting the damage of dye from the UV light and singlet oxygen.

The other aim of this invention is to provide a kind of cyanine-TCNQ complex dyes mixture (II, III, and IV), used for the data storage media, having high oxidation potential to prevent the oxidation of dyes.

The other aim of this invention is to provide a kind of cyanine-TCNQ complex dyes mixture (II, III, and IV), used for the data storage media, in which they can adjust the primary absorption position with wavelength in the range of 400˜800 nm and possess high fluorescent quantum efficiency.

The other aim of this invention is to provide a kind of cyanine-TCNQ complex dyes mixture (II, III, and IV), used for the data storage media, in which it is not necessary to add any photostabilizing agent since these dyes possess the excellent photostability. (photo-endurance: 3000 W Xe lamp irradiates for 1200 min., CNR≧35 dB, the change of reflection index for an optical disc with wavelength at 635 nm is smaller than 1%.)

The other aim of this invention is to provide a kind of cyanine-TCNQ complex dyes mixture (II, III, and IV), used for the data storage media, in which the manufacture cost can be reduced since it is not necessary to add any photostabilizing agent.

The other aim of this invention is to provide a kind of cyanine-TCNQ complex dyes mixture (II, III, and IV), used for the data storage media, having the better solubility for organic solvents.

In order to get the aims described above, this invention is to provide a kind of cyanine-TCNQ complex dyes mixture (II, III, and IV), used for the data storage media, having the structural formula (I) as shown in the following:

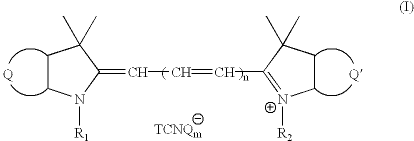

wherein Q and Q′ denote aromatic or polyaromatic, R1 and R2 denote one of alkyl, arylester, alkoxy, alkylthio, and alkoxythio etc., n represents an integer of 0, 1, 2, and 3, TCNQ-m represents 7,7′,8,8′-tetracyanoquinodimethane and its derivatives, and m represents an integer of 1 or 2. These dyes have the extremely large absorption and high fluorescent quantum efficiency in the visible light region with λ=400 nm˜800 nm, and can be used for the recording layer materials of the reflection optical recording media and the non-reflection fluorescent optical recording media after the suitable formulation.

### BRIEF DESCRIPTION OF THE DRAWINGS

FIG. 1 illustrates the basic skeleton of the structural formula for cyanine-TCNQ complex dye in this invention.

FIG. 2 illustrates the structural formula for cyanine-TCNQ complex dye (II) in this invention.

FIG. 3 illustrates the structural formula for the compound abbreviated as MIB in the synthetic example of this invention.

FIG. 4 illustrates the structural formula for the compound abbreviated as BII in the synthetic example of this invention.

FIG. 5 illustrates the structural formula for the compound abbreviated as MBTI in the synthetic example of this invention.

FIG. 6 illustrates the structural formula for the reaction intermediate (A) in the synthetic example of this invention.

FIG. 7 illustrates the UV/Visible/IR absorption spectrum of the cyanine-TCNQ complex dye (II) in this invention.

FIG. 8 illustrates an infrared (IR) spectrum of the cyanine-TCNQ complex dye (II) in this invention.

FIG. 9 illustrates the thermoweighing spectrum (10° C./min) of the cyanine-TCNQ complex dye (II) in this invention.

FIG. 10 illustrates the structural formula for cyanine-TCNQ complex dye (III) in this invention.

FIG. 11 illustrates the UV/Visible/IR absorption spectrum of the cyanine-TCNQ complex dye (III) in this invention.

FIG. 12 illustrates an infrared (IR) spectrum of the cyanine-TCNQ complex dye (III) in this invention.

FIG. 13 illustrates the thermoweighing spectrum (10° C./min) of the cyanine-TCNQ complex dye (III) in this invention.

FIG. 14 illustrates the structural formula for cyanine-TCNQ complex dye (IV) in this invention.

FIG. 15 illustrates the UV/Visible/IR absorption spectrum of the cyanine-TCNQ complex dye (IV) in this invention.

FIG. 16 illustrates an infrared (IR) spectrum of the cyanine-TCNQ complex dye (IV) in this invention.

FIG. 17 illustrates the thermoweighing spectrum (10° C./min) of the cyanine-TCNQ complex dye (IV) in this invention.

FIG. 18 illustrates the UV/Visible/IR absorption spectrum of the mixture of the cyanine-TCNQ complex dye (III) and the cyanine-TCNQ complex dye (IV) coated on the blank substrate plate in this invention.

FIG. 19 illustrates the UV/Visible/IR reflection spectrum of the silver reflection layer manufactured with a mixture of the cyanine-TCNQ complex dye (III) and the cyanine-TCNQ complex dye (IV) in this invention.

FIG. 20 illustrates the disc reading-writing test analysis and photo-endurance test experiment for an optical disc manufactured with a mixture of the cyanine-TCNQ complex dye (III) and the cyanine-TCNQ complex dye (IV) in this invention.

FIG. 21 illustrates the reflection index spectrum and photo-endurance test experiment for an optical disc manufactured with a mixture of the cyanine-TCNQ complex dye (III) and the cyanine-TCNQ complex dye (IV) in this invention.

FIG. 22 illustrates the fluorescent spectrum irradiated at 551 nm of the cyanine-TCNQ complex dye (III) and the fluorescent photo of its thin layer in this invention.

### DETAILED DESCRIPTION OF THE PREFERRED EMBODIMENTS

This invention is to combine the TCNQ molecule and cyanine dye to form a complex, in which its structure possesses both photostability and good solubility of organic solvents.

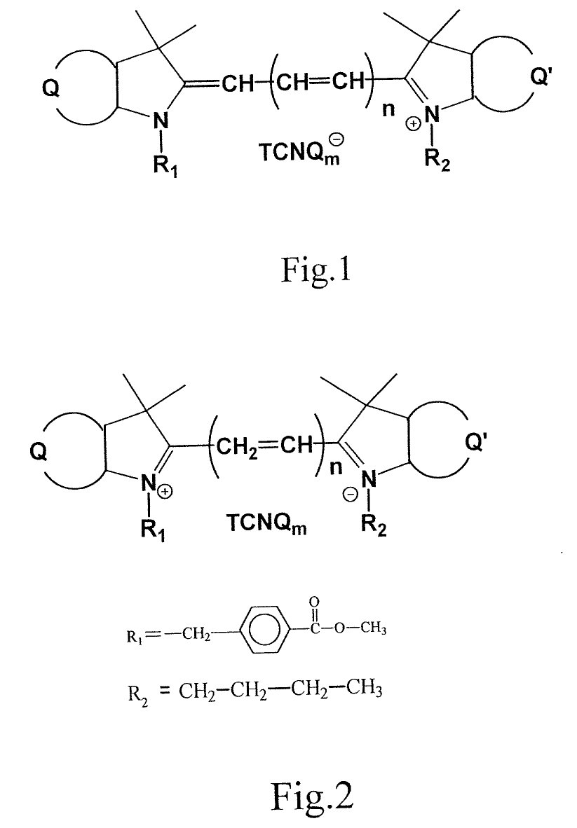

The structural formula (I) of cyanine-TCNQ complex dye in this invention is shown as FIG. 1, wherein Q and Q′ are selected from one of aromatic and polyaromatic, R1 and R2 are selected from the group consisting of alkyl, arylester, alkoxy, alkylthio, and alkoxythio etc., n represents an integer of 0, 1, 2, and 3, TCNQ-m represents 7,7′,8,8′-tetracyanoquinodimethane or its derivatives, and m represents an integer of 1 or 2.

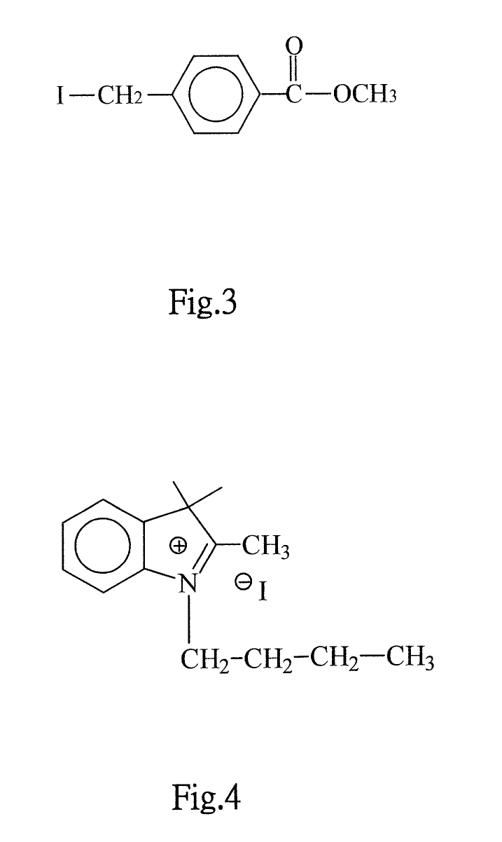

FIG. 2 shows the cyanine-TCNQ dye (II) wherein n=1, R1 is —CH₂C₆H₄COOCH₃, R2 is n-butyl group; FIG. 3 shows the cyanine-TCNQ dye (III) wherein n=1, both R1 and R2 are —CH₂C₆H₄COOCH₃; the cyanine-TCNQ dye (IV) wherein n=2, both R1 and R2 are —CH₂C₆H₄COOCH₃.

This invention is to bond the TCNQ molecule on cyanine dye to form a stable charge transfer complex. It has a nice absorption both in UV light region with wavelength at 200 nm˜400 nm and in near IR light region with 800 nm˜1000 nm; and it remove the short wave light resource (the wavelength is smaller than 400 nm), which possesses the ability to break the chemical bonding, through the charge transfer and irradiation of fluorescent light pathway to transfer the energy. In addition, since the cyanine TCNQ charge transfer complex possesses the higher oxidation potential than that of general cyanine halide, cyanine ClO₄, and cyanine PF₆ complex, it means that TCNQ possesses the better inhibition of singlet oxygen attacking the main part of cyanine dye, and it can increase the photostability of cyanine dye to solve the problem of the addition of photostabilizing agent in cyanine dye. In this invention the experimental example is the cyanine dye as the description example.

In this invention the manufactured method of the described new type of cyanine TCNQ complex dye is firstly to synthesize the substituted methyl (4-iodomethyl) benzoate, which was obtained from the reaction of 4-chloromethyl benzoyl chloride with methanol in benzene containing pyridine heated at 40° C.-60° C. for several hours and then displaced of chloride with sodium iodide. Reaction of the substituted methyl (4-iodomethyl) benzoate with 2,3,3-trimethyl-4,5-benzo-3H-indole in benzene heated at 80° C.˜100° C., after 3˜8 hours, 1-(4′-methoxycarbonyl)benzyl-2,3,3-trimethyl-4,5-benzo-3H-indolium iodide was obtained. Afterwards, 1-butyl-2,3,3-trimethyl indolenine was synthesized by the reaction of iodobutane with 2,3,3-trimethyl indolenine in benzene heated at 80° C.˜100° C. for 3˜8 hours. Afterwards, reaction of 1-(4′-methoxy-carbonyl)-benzyl-2,3,3-trimethyl-4,5-benzo-3H-indolium iodide with N,N′-diphenylformamidine in acetic anhydride heated together at 100° C.˜120° C. for 1˜3 hours, the compound (A) was obtained. Afterwards, a mixture of 1-butanyl-2,3,3- trimethylindolenine, compound (A), and sodium acetate in acetic anhydride were heated at 100° C.˜120° C. for 1˜3 hours, then displaced of iodide with LiTCNQ (the synthesis of LiTCNQ is according to L. R. Melby et al., J. Am. Chem. Soc., 84, p.3374, (1962)) to obtain the compound (B), 2-[3-(1,3-dihydro-1,1-dimethyl-3-(4′-methoxycarbonyl)-benzyl-2H-benze[e]indol-2-ylidene)-1-propenyl]1,1 -dimethyl-3-butyl-1H indolium TCNQ.

In this invention the synthesized new type of cyanine TCNQ complex dyes are soluble in alcohols (e.g. methanol, ethanol, and isopropyl alcohol etc.), ketones (e.g. acetone and methyl ethyl ketone (MEK) etc.), ethers (e.g. diethyl ether, and tetrahydrofuran etc.), chloroform, dichloromethane, and DMF etc. organic solvents. Therefore, it can utilize spurt coating, roll-pressure coating, dip or spin coating etc. manufacture method to produce the containing cyanine TCNQ complex dye thin layer. It is primary to bond the TNCQ molecule on cyanine dye to form a stable complex, since cyanine TCNQ complex possesses the higher oxidation potential than that of general cyanine halide, cyanine ClO₄, and cyanine PF₆ complex, it means that TCNQ possesses the better inhibition of singlet oxygen attacking the main part of cyanine dye, and it can increase the photostability of cyanine dye to solve the problem of the addition of photostabilizing agent in cyanine dye.

In this invention the new type of cyanine TCNQ complex dye not only can be used as optical recording media material, but also utilized as the increasing sensitizer for silver salt photo, copy writing and printing.

In order to understand clearly the described aims above, other objectives, characteristics, and advantages of this invention, it describes this invention in details with the following experimental examples and applied examples as well as the accompanying figures. It describes in details for the preparation the new type of cyanine TCNQ complex dye in this invention using the experimental example as follows.

### EXPERIMENTALS

### Experimental Example 1

### Preparation Example

Preparation of 2-[3-(1,3-dihydro-1,1-dimethyl-3(4′-methoxycarbonyl)-benzyl-2H-benze[e]indol-2-ylidene)-1-propenyl]1,1-dimethyl-3-butyl-1H indolium TCNQ (abbreviated as SL-TCNQ), SL cyanine TCNQ dye, the structural formula of cyanine TCNQ complex dye (II) as shown in FIG. 2, needs through the following described procedure:

(a) Preparation of Methyl (4-iodomethyl) Benzoate (Abbreviated as MIB), the Structural Formula of MIB as Shown in FIG. 3

A mixed solution of 4-chloromethylbenzoyl chloride (TCI Chemical) (1.89 g), methanol (0.32 g), and pyridine (0.791 g) in benzene (25 mL) was heated at 45° C. for 1˜3 hours, filtered and evaporated to obtain white solids. Dissolved white solids in acetone and added sodium iodide (1.50 g), a mixture was heated at 40° C. for 3˜5 hours, filtered, extracted from dichloromethane and water, evaporated to give the light yellow solids. The yield was 90%, m.p. 67° C.

(b) Preparation of 1-butyl-2,3,3-trimethyl Indoleninium Iodide (Abbreviated as BII), the Structural Formula of BII as Shown in FIG. 4.

A mixed solution of iodobutane (TCI Chemical) (2.76 g) and 2,3,3-trimethyl indolenine (TCI Chemical) (2.38 g) in benzene was heated at 80° C.˜85° C. for 4 hours. The solution was evaporated and recrystallized from ethyl acetate to give light yellow crystals. The yield was 85%, m.p. 102° C.

(c) Preparation of 1-(4′-methoxycarbonyl)benzyl-2,3,3-trimethyl-4,5-benzo-3H-indoleninium Iodide (Abbreviated as MBTI), the Structural Formula of MBTI as Shown in FIG. 5

A mixed solution of MIB (2.76 g) and 2,3,3-trimethyl-4,5-benzo-3H-indole (TCI Chemical) (1.75 g) in benzene was heated at 80° C.˜90° C. for 4˜8 hours. The solution was evaporated and recrystallized from methanol to give light grey crystals. The yield was 75%, m.p. 120° C.

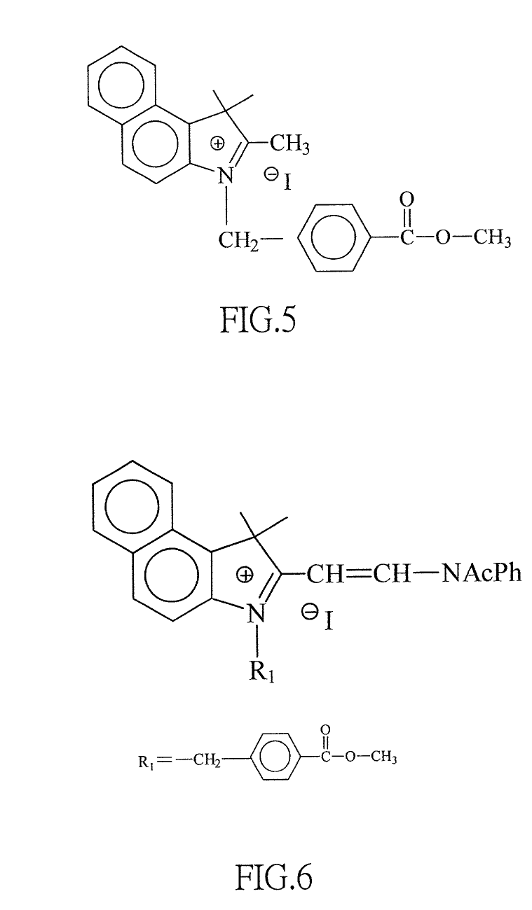

(d) Preparation of Intermediate (A), the Structural Formula of (A) as Shown in FIG. 6

A mixed solution of MBTI (6.16 g) and N,N′-diphenylformamidine (1.12 g) in acetic anhydride (20 mL) was heated at 100° C.˜120° C. for 2 hours. After cooling, the reaction mixture was poured into an aqueous solution of sodium perchlorate (1.41 g) to give solid, then was dissolved in dichloromethane, washed with water several times, evaporated and recrystallized from ethanol to give intermediate (A). The yield was 70%.

(e) Preparation of 2-[3-(1,3-dihyhro-1,1-dimethyl-3-(4′-methoxycar-bonyl)-benzyl-2H-benze[e]indol-2-ylidene)-1-propenyl]1,1-dimethyl-3-butyl-1H Indolium TCNQ (II), the Structural Formula of Cyanine TCNQ Complex Dye (II) as Shown in FIG. 2

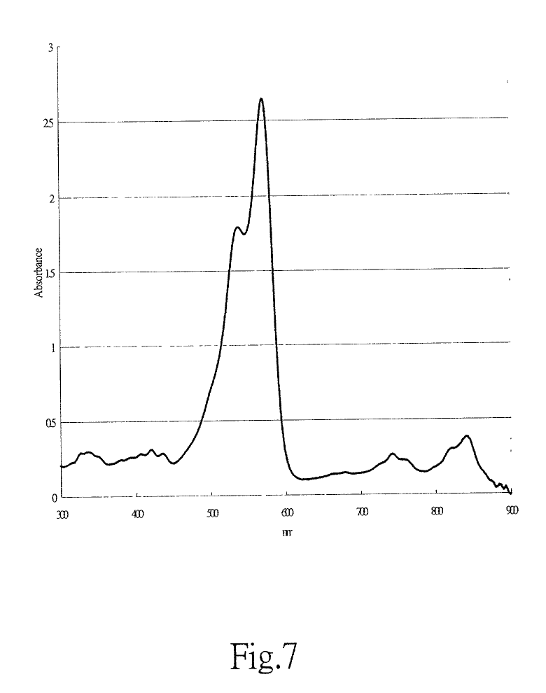

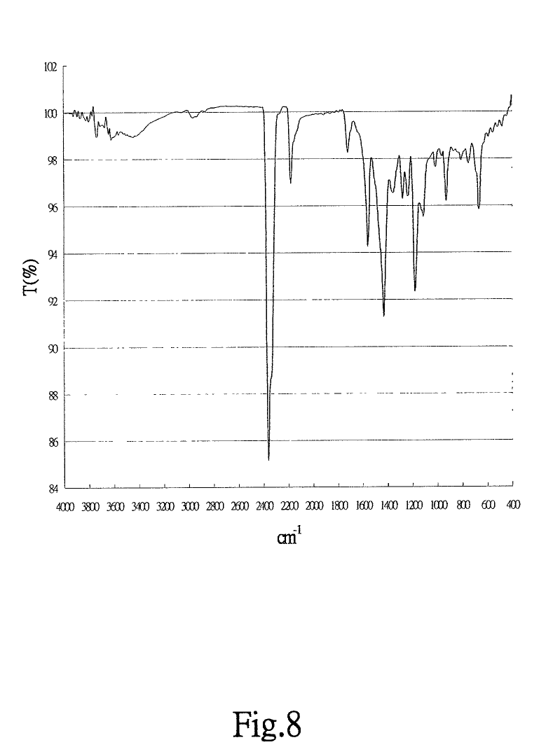

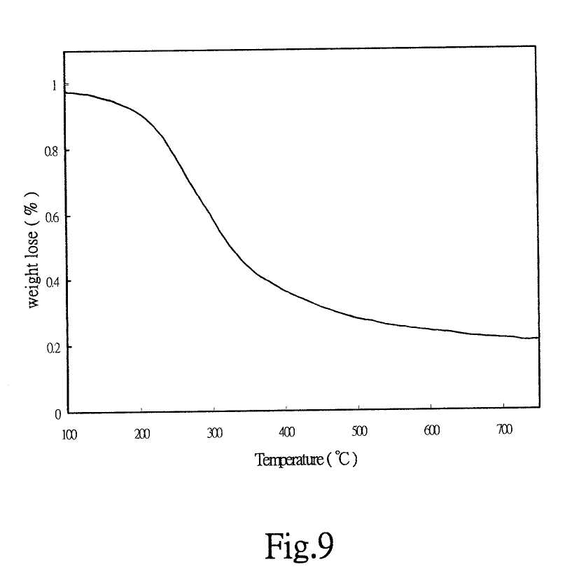

Placed 1-(4′-methoxycarbonyl)-2,3,3-trimethyl-4,5-benzo-3H-indole (0.02 mole) and ethyl orthoformate (TCI Chemical) (0.01 mole) in a two-necked flask, dissolved with pyridine, then heated at 110° C.˜120° C. for 1˜3 hours. After cooling, the reaction mixture was poured into an aqueous solution of LiTCNQ (1.24 g) to give solid, recrystallized from methanol to give dark red crystals (III). The yield was 70%, m.p. 190° C. FIG. 7 shows that the UV/Visible/IR absorption spectrum for cyanine TCNQ complex dye (II), □max (EtOH)=568 nm; FIG. 8 and FIG. 9 show that IR spectrum and thermoweighing spectrum(10° C./min) for cyanine TCNQ complex dye (II), respectively.

### Experimental Example 2

### Preparation Example

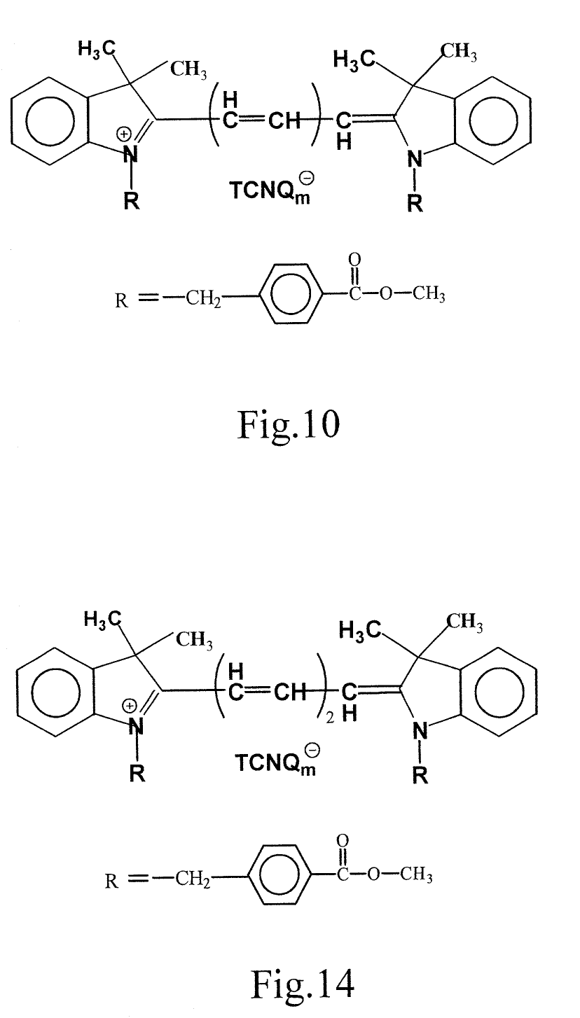

Preparation of 1-(4′-methoxycarbonyl)benzyl-3,3 -trimethyl-1′-(4′-methoxycarbonyl) benzyl-3,3′-dimethylindo-2,2′-trimethine TCNQ (abbreviated as S-TCNQ), S cyanine TCNQ dye, the structural formula of cyanine TCNQ complex dye (III) as shown in FIG. 10, needs through the following described procedure:

(a) Preparation of Methyl (4-iodomethyl) Benzoate (MIB)

First, a mixed solution of 4-chloromethylbenzoyl chloride (TCI Chemical) (1.89 g, 0.01 mole), methanol (0.32 g, 0.01mole), and pyridine (0.791 g, 0.01 mole) in benzene was heated at 40° C. for 3 hours, then filtered and evaporated to obtain white solids. Dissolved white solids in acetone and added sodium iodide (1.50 g), a mixture was heated at 40° C. for 3 hours, filtered, extracted from dichloromethane and water, evaporated to give the light yellow products.

(b) Preparation of 1-(4′-methoxycarbonyl)benzyl-2,3,3 -trimethyl-4,5-benzo- 3H-indole

A mixed solution of MIB (0.01 mole) and 2,3,3-trimethyl-4,5-benzo-3H-indole (TCI Chemical) (0.01 mole) in MEK was heated at 80° C. for 3 hours. The solution was evaporated and recrystallized from ethyl acetate to give white crystals.

(c) Preparation of 1-(4′-methoxycarbonyl)benzyl-2,3,3-trimethyl-1′-(4′-methoxycarbonyl) benzyl-3,3 ′-dimethylindo-2,2′-trimethine TCNQ

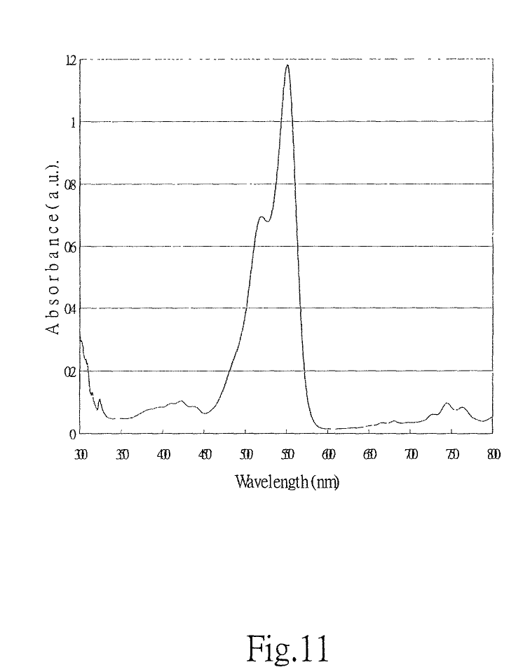

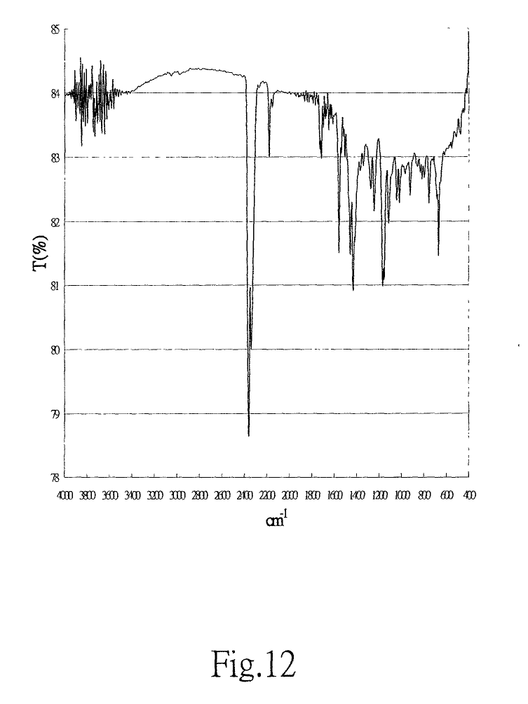

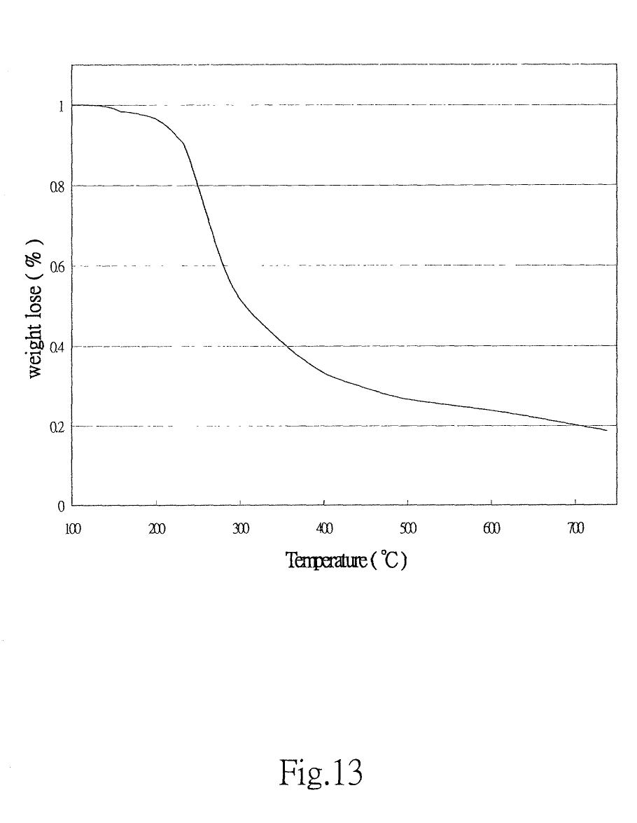

A mixed solution of 1-(4′-methoxycarbonyl)benzyl-2,3,3-trimethyl-4,5-benzo-3H-indole (0.02mole) and ethyl orthoformate (TCI Chemical) (0.01 mole) in pyridine was heated at 110° C.˜120° C. for 1˜3 hours. After cooling, the reaction mixture was poured into an aqueous solution of LiTCNQ (1.24g) to give solid, recrystallized from methanol to give dark red crystals (III). The yield was 73%, m.p. 178° C. FIG. 11 shows that the UV/Visible/IR absorption spectrum for cyanine TCNQ complex dye (III), □max (EtOH)=551 nm; FIG. 12 and FIG. 13 show that IR spectrum and thermoweighing spectrum (10° C./min) for cyanine TCNQ complex dye (III), respectively.

### Experimental Example 3

### Preparation Example

Preparation of 1-(4″-methoxycarbonyl)benzyl-3,3-dimethyl- 1′-(4″-methoxycarbonyl) benzyl-3,3′-dimethylindo-2,2′-pentamethine TCNQ, the structural formula of cyanine TCNQ complex dye (IV) as shown in FIG. 14, needs through the following described procedure:

(a) Preparation of the substituted methyl (4-iodomethyl) benzoate first, then preparation of 1-1-(4′-methoxycarbonyl)benzyl-2,3,3-trimethylindoleninium iodide, finally reaction with 3-anilinoacryl-aldehyde anil gave the structural formula (IV) dye.

(b) Preparation of Methyl (4-iodomethyl) Benzoate (MIB), the Structural Formula of MIB as Shown in FIG. 3

A mixed solution of 4-chloromethylbenzoyl chloride (TCI Chemical) (1.89 g), methanol (0.32 g), and pyridine (0.791 g) in benzene (25 mL) was heated at 40° C. for 3 hours, filtered and evaporated to obtain white solids. Dissolved white solids in acetone and added sodium iodide (1.50 g), a mixture was heated at 40° C. for 3 hours, filtered, extracted from dichloromethane and water, evaporated to give the light yellow solids. The yield was 90%, m.p. 67° C.

(c) Preparation of 1-(4′-methoxycarbonyl)benzyl-2,3,3-trimethyl-4,5-benzo- 3H-indoleninium Iodide (MBTI), the Structural Formula of MBTI as Shown in FIG. 5

A mixed solution of MIB (2.76 g) and 2,3,3-trimethyl-4,5-benzo-3H-indolenine (TCI Chemical) (1.75 g) in benzene was heated at 80° C.˜85° C. for 6 hours. The solution was evaporated and recrystallized from ethyl acetate to give light yellow crystals. The yield was 71%, m.p. 109° C.

(d) Preparation of 1-(4″-methoxycarbonyl)benzyl-3,3-dimethyl-1′-(4″-methoxycarbonyl)-benzyl-3′,3′-dimethylindo-2,2′-pentamethine TCNQ (IV)

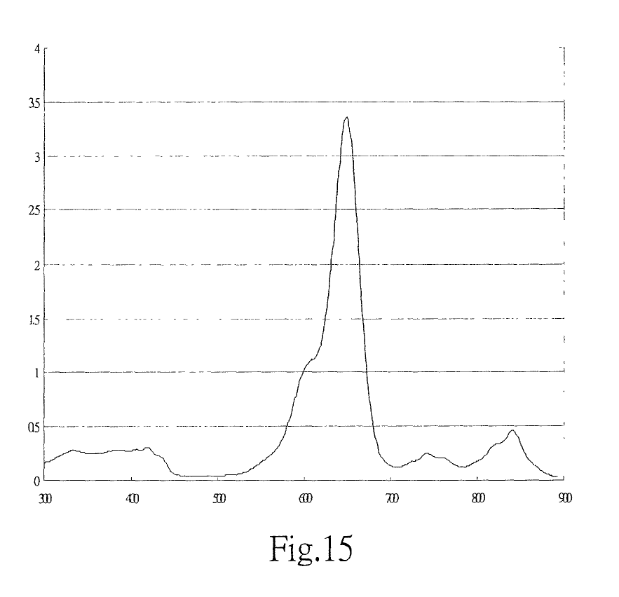

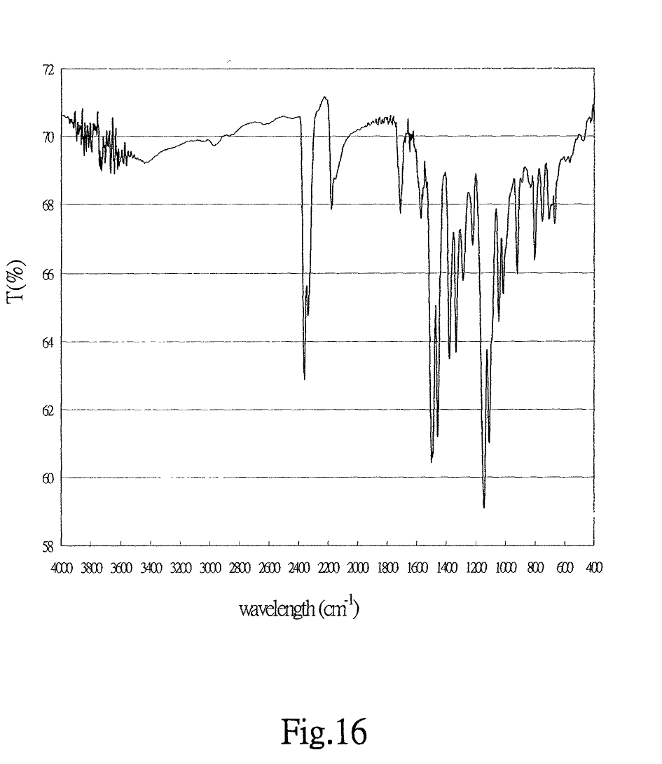

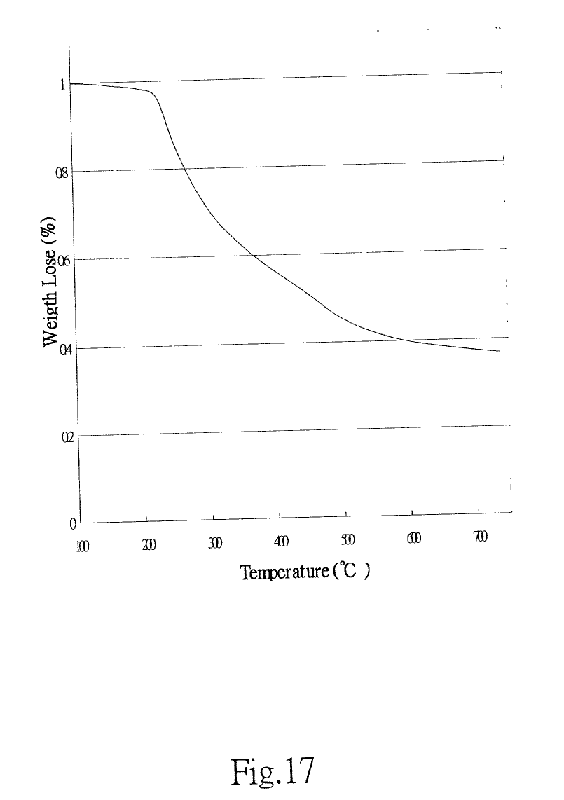

A mixed solution of 1-(4′-methoxycarbonyl)benzyl-2,3,3-trimethylindoleninium iodide (6.16 g), 3-anilinoacrylaldehyde anil (TCI Chemical) (2.85 g) and sodium acetate (1.64 g) in acetic anhydride (20mL) was heated at 100° C. for 2 hours. After cooling, the reaction mixture was poured into an aqueous solution of LiTCNQ (1.41 g) to give solid, which was dissolved in dichloromethane and washed with water several times, evaporated and recrystallized from ethanol to give dark green crystals (IV). The yield was 70%, m.p. 201° C. FIG. 15 shows that the UV/Visible/IR absorption spectrum for cyanine TCNQ complex dye (IV), □max (EtOH)=649 nm; FIG. 16 and FIG. 17 show that IR spectrum and thermoweighing spectrum (10° C./min) for cyanine TCNQ complex dye (IV), respectively.

### Experimental Example 4

### Applied Example

The Manufacture Process of Recordable Optical Disc

New type of cyanine TCNQ complex dye (II) (1.50 g) and cyanine dye (IV) (0.075 g) were dissolved in 2,2,3,3-tetrafluoropropanol to form a 100 g solution. The dyes were coated onto blank substrate plate by a spin coater, the whole coating processes of a coater are as follows: a dipping process: at 30˜500 rpm, 2˜10 seconds; a spinning out process: at 1000˜3000 rpm, 10˜30 seconds; and a baking process: at 2000˜5000 rpm, 10˜30 seconds. The recording layer, coated by TCNQ dye onto the substrate plate, has the thickness of about 500 Å˜2000 Å, on which it was then sputtered with a layer of Au, Ag, Al, Cu, Cr and its alloy to form the reflection layer having a thickness of about 500 Å˜1000 Å; finally, combination of a substrate plate sputtered with a reflection layer and a recording layer with the other PC blank substrate plate (e.g. DVD-R 0.6mm substrate plate) together (combination method: spin coating method, screen printing method, and thermomelting glue method etc.) forms a piece of high density recordable optical disc. Using Weather-Ometer (ATLAS Ci400) to provide a wavelength at 420 nm and 3000 W of Xenon lamp to irradiate the thin recording layer causes the damage of the thin recording layer dye. Using optical density spectrometer (ETA-OD) and Pulstec DDU 1000 measure the thin layer reflection spectrum, respectively; the value of 3T CNR is changed from according to the Xenon lamp irradiation time.

While it is preparing the data storage media by using this invention cyanine TCNQ dye (II) and cyanine TCNQ dye (IV), the weight percentage of cyanine TCNQ dye (IV) to cyanine TCNQ dye (II) can be 0.5%˜20%, and preferably 2%˜10%. However, the weight percentage of cyanine TCNQ dye (II) to the total solution (including cyanine TCNQ dye (II), cyanine TCNQ dye (IV), and solvent) can be 0.5%˜10 %, and preferably 1%˜5%.

While it is preparing the data storage media by using this invention cyanine TCNQ dye (III) and cyanine TCNQ dye (IV), the weight percentage of cyanine TCNQ dye (IV) to cyanine TCNQ dye (III) can be 0.5%˜20%, and preferably 2%˜10%. However, the weight percentage of cyanine TCNQ dye (III) to the total solution (including cyanine TCNQ dye (III), cyanine TCNQ dye (IV), and solvent) can be 0.5%˜10%, and preferably 1%˜5%.

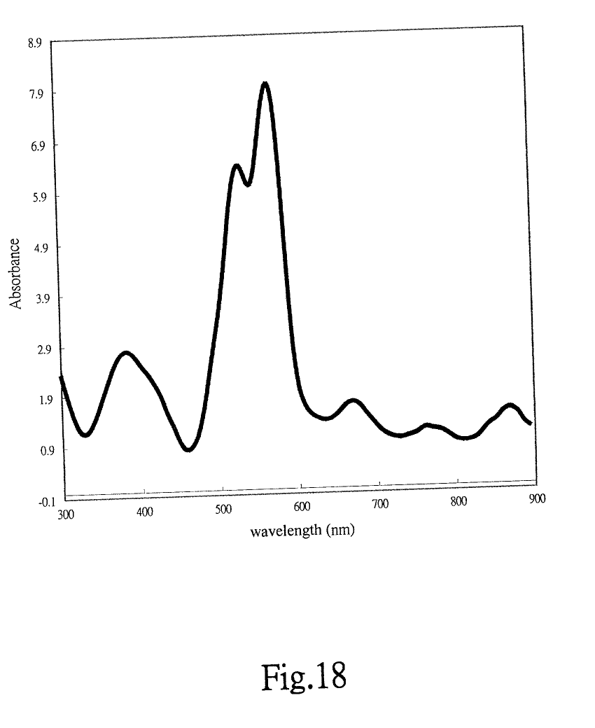

While it is preparing the data storage media by using this invention cyanine TCNQ complex dyes, the organic solvent can be selected one of from the following compound: 2,2,3,3-tetrafluoropropanol, alcohol, ketone, ether, chloroform, dichloromethane, and DMF etc. FIG. 18 shows the UV/Visible/IR absorption spectrum for the combination of cyanine TCNQ complex dye (III) and cyanine TCNQ complex dye (IV) coated onto the blank substrate plate.

These kind of compounds have nice absorptions both in UV light region with wavelength at 200 nm˜400 nm and in near IR light region with 800 nm˜1000 nm; and they can remove the short wave light resource (the wavelength is smaller than 400 nm), which possesses the ability to break the chemical bonding, through the charge transfer and irradiation of fluorescent light pathway to transfer the energy. In addition, since these cyanine TCNQ dyes possess the higher oxidation potential than that of general cyanine dyes, it means that TCNQ possesses the better inhibition of singlet oxygen attacking the main part of cyanine dye. Therefore, dyes possess the excellent photostability in the condition of no addition of photostabilizing agent.

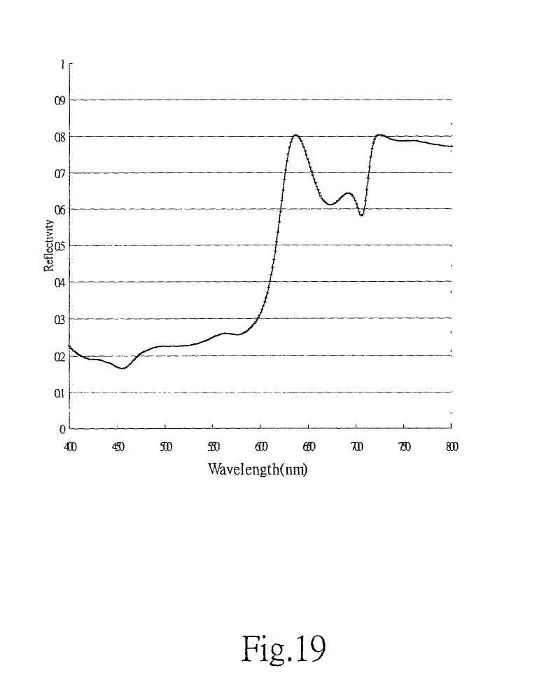

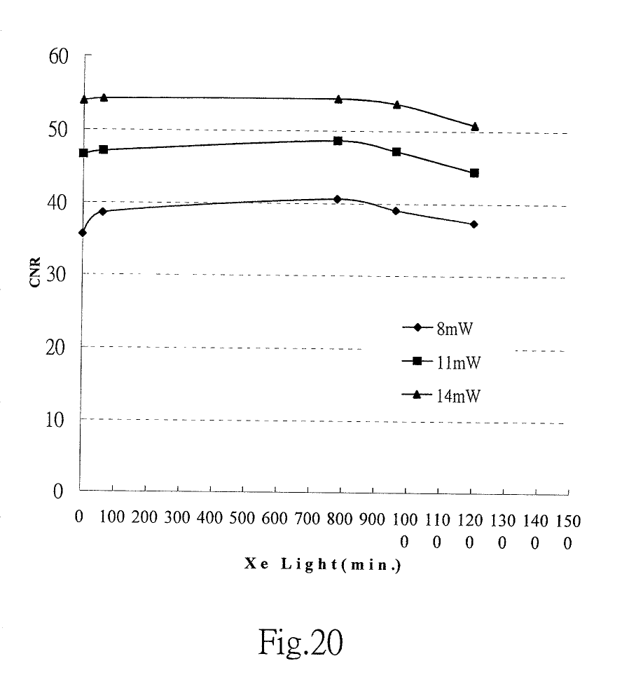

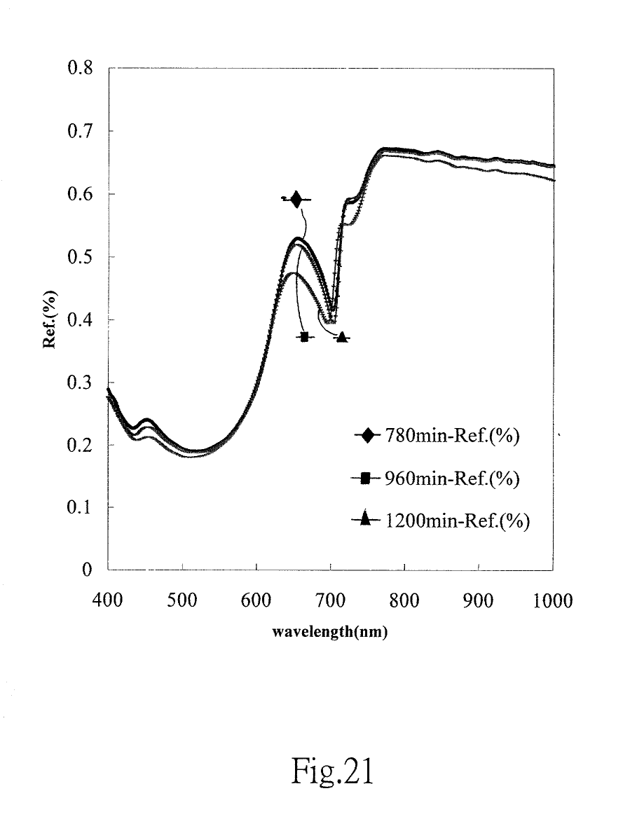

FIG. 19 shows the UV/Visible/IR reflection spectrum for the silver reflection layer of optical disc manufactured by the combination of cyanine TCNQ complex dye (III) and cyanine TCNQ complex dye (IV). This kind structure of high density recordable optical disc has reflection index above 60% with λ=635 nm measured from the spectrum of reflection index vs. wavelength. FIG. 20 shows the disc reading-writing examination analysis of the photo-endurance examination test for the optical disc manufactured by the combination of cyanine TCNQ complex dye (III) and cyanine TCNQ complex dye (IV). FIG. 21 shows the reflection index spectrum of the photo-endurance examination test for the optical disc manufactured by the combination of cyanine TCNQ complex dye (III) and cyanine TCNQ complex dye (IV). It shows that cyanine TCNQ complex dyes of this invention can be successfully applied to the high density of optical disc storage system, and it is not necessary to add any photostabilizing agent to possess the excellent photo-endurane.

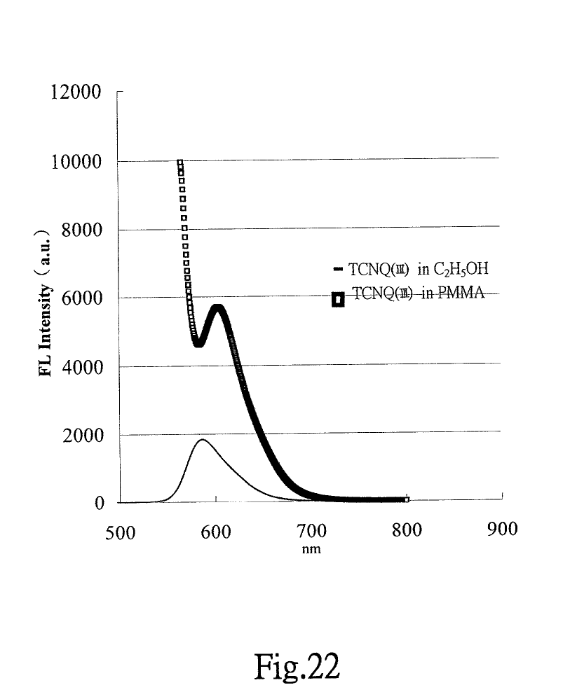

FIG. 22 shows the fluorescent spectrum obtained from cyanine TCNQ complex dye (III) with 551 nm of the irradiating light source while it is preparing the data storage media by using cyanine TCNQ complex dye (III). The fluorescent recording layer components consist of at least cyanine TCNQ complex dye, polymer resin, and interfacial glue, but not containing photostabilizing agent.

This invention specially discloses and describes selected the best examples. It is to be understood, however, that this invention is not limited to the specific features shown and described. The invention is claimed in any forms or modifications within the spirit and the scope of the appended claims.

What is claimed is:

1. A kind of cyanine-TCNQ complex dyes mixture (II, III, and IV) used as the data storage media having the structural formula (I) as follows: 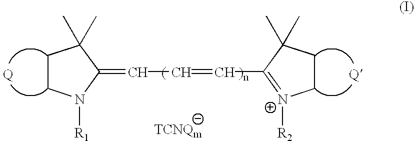
wherein Q and Q′ are selected from one of aromatic and polyaromatic, R1 and R2 are selected from the group consisting of alkyl, arylester, alkoxy, alkylthio, and alkoxythio etc., n represents an integer of 0, 1, 2, and 3, TCNQ-m represents 7,7′,8,8′-tetracyanoquinodimethane and its derivatives, and m represents an integer of 1 or 2.

2. A cyanine TCNQ complex dye used for the data storage media of claim 1, wherein said in cyanine TCNQ dye (I), n represents an integer of 0, 1, 2, and 3, R1 is —CH₂C₆H₄COOCH₃, R2 is linear alkyl group (carbon number is C1˜C18), and m represents an integer of 1 or 2.

3. A cyanine TCNQ complex dye used for the data storage media of claim 1, wherein said in cyanine TCNQ dye (I), n represents an integer of 0, 1, 2, and 3, R1 and R2 are both of —CH₂C₆H₄COOCH₃, and m represents an integer of 1 or 2.

4. A cyanine TCNQ complex dye used for the data storage media of claim 1, wherein said in cyanine TCNQ dye (II), n=1, R1 is —CH₂C₆H₄COOCH₃, R2 is linear butyl group; in cyanine TCNQ dye (III), n=1, R1 and R2 are both of —CH₂C₆H₄COOCH₃; in cyanine TCNQ dye (IV), n=2, R1 and R2 are both of —CH₂C₆H₄COOCH₃.

5. A cyanine TCNQ complex dye used for the data storage media of claim 1, wherein said while it is preparing the data storage media by using this cyanine TCNQ complex dye, the reflection layer is selected from the group consisting of Au, Ag, Al, Cu, Cr, and its alloy.

6. A cyanine TCNQ complex dye used for the data storage media of claim 1, wherein said while it is preparing the data storage media by using this cyanine TCNQ complex dye obtained from the combination of cyanine TCNQ dye (II) and cyanine TCNQ dye (IV), and the weight percentage of cyanine TCNQ dye (IV) to TCNQ dye (II) can be 0.5%˜20%.

7. A cyanine TCNQ complex dye used for the data storage media of claim 1, wherein said while it is preparing the data storage media by using this cyanine TCNQ complex dye obtained from the combination of cyanine TCNQ dye (II) and cyanine TCNQ dye (IV), and the weight percentage of cyanine TCNQ dye (IV) to TCNQ dye (II) can preferably be 2%˜0%.

8. A cyanine TCNQ complex dye used for the data storage media of claim 1, wherein said while it is preparing the data storage media by using this cyanine TCNQ complex dye obtained from the combination of cyanine TCNQ dye (II) and cyanine TCNQ dye (IV), and the weight percentage of cyanine TCNQ dye (II) to the total solution (including TCNQ dye (II), TCNQ dye (IV), and solvent) can be 0.5%˜10%.

9. A cyanine TCNQ complex dye used for the data storage media of claim 1, wherein said while it is preparing the data storage media by using this cyanine TCNQ complex dye obtained from the combination of cyanine TCNQ dye (II) and cyanine TCNQ dye (IV), and the weight percentage of cyanine TCNQ dye (II) to the total solution (including TCNQ dye (II), TCNQ dye (IV), and solvent) can preferably be 1%˜5%.

10. A cyanine TCNQ complex dye used for the data storage media of claim 1, wherein said while it is preparing the data storage media by using this cyanine TCNQ complex dye obtained from the combination of cyanine TCNQ dye (III) and cyanine TCNQ dye (IV), and the weight percentage of cyanine TCNQ dye (IV) to TCNQ dye (III) can be 0.5%˜20%.

11. A cyanine TCNQ complex dye used for the data storage media of claim 1, wherein said while it is preparing the data storage media by using this cyanine TCNQ complex dye obtained from the combination of cyanine TCNQ dye (III) and cyanine TCNQ dye (IV), and the weight percentage of cyanine TCNQ dye (IV) to TCNQ dye (III) can preferably be 2%˜10%.

12. A cyanine TCNQ complex dye used for the data storage media of claim 1, wherein said while it is preparing the data storage media by using this cyanine TCNQ complex dye obtained from the combination of cyanine TCNQ dye (III) and cyanine TCNQ dye (IV), and the weight percentage of cyanine TCNQ dye (III) to the total solution (including TCNQ dye (III), TCNQ dye (IV), and solvent) can be 0.5%˜10%.

13. A cyanine TCNQ complex dye used for the data storage media of claim 1, wherein said while it is preparing the data storage media by using this cyanine TCNQ complex dye obtained from the combination of cyanine TCNQ dye (III) and cyanine TCNQ dye (IV), and the weight percentage of cyanine TCNQ dye (III) to the total solution (including TCNQ dye (III), TCNQ dye (IV), and solvent) can preferably be 1%˜5%

14. A cyanine TCNQ complex dye used for the data storage media of claim 1, wherein said while it is preparing the data storage media by using this cyanine TCNQ complex dye, the organic solvent can be selected one of from the following compound: 2,2,3,3-tetrafluoropropanol, alcohol, ketone, ether, chloroform, dichloromethane, and DMF etc.

15. A cyanine TCNQ complex dye used for the data storage media of claim 1, wherein said while it is preparing the data storage media by using this cyanine TCNQ complex dye, the fluorescent recording layer components consist of at least cyanine TCNQ complex dye, polymer resin, and interfacial glue.

16. A cyanine TCNQ complex dye used for the data storage media of claim 1, wherein said while it is preparing the data storage media by using this cyanine TCNQ complex dye, the fluorescent recording layer does not contain any photostabilizing agent.
